(19) United States
(12) Patent Application Publication
(10) Pub. No.: US 2024/0049717 A1
(43) Pub. Date: Feb. 15, 2024

(54) SYSTEM AND METHOD FOR SILVER NANOPARTICLES

(71) Applicant: RAFIQUL ISLAM, Tempe, AZ (US)

(72) Inventors: RAFIQUL ISLAM, GILBERT, AZ (US); Dianne Carrillo, Gilbert, AZ (US); Yuqiang Bi, GILBERT, AZ (US); ALI ANSARI, GILBERT, AZ (US)

(21) Appl. No.: 18/376,985

(22) Filed: Oct. 5, 2023

Related U.S. Application Data
(63) Continuation-in-part of application No. PCT/US2021/035410, filed on Jun. 2, 2021.

(51) Int. Cl. A01N 59/16; A01N 25/26; A01P 1/00; B82Y 5/00; B82Y 40/00; B22F 1/16; B22F 1/054; B22F 9/24; B22F 1/142

(52) U.S. Cl. CPC A01N 59/16; A01N 25/26; A01P 1/00; B82Y 5/00; B82Y 40/00; B22F 1/16; B22F 1/056; B22F 9/24; B22F 1/142; B22F 2999/00; B22F 2998/10; B22F 2304/054; B22F 2304/058; B22F 2301/255; B22F 2302/45

(57) ABSTRACT

The present invention provides among other things silver nanoparticles and methods of making the same. The nanoparticles may be sulfidated to decrease the silver leaching rate and sustain the biocidal properties. Such nanoparticles may be applied as a coating or additive to substrates such as metals, alloys, polymers, membranes, textiles, and other such materials, allowing for the substrates to exhibit antimicrobial properties.

### CROSS REFERENCE TO RELATED APPLICATIONS

The present application is a continuation-in-part application of PCT Application No. PCT/US21/35410, filed on Jun. 2, 2021, entitled “Passivated Silver Nanoparticle Coatings and Methods of Making the Same,” which claims priority to U.S. Provisional Application No. 63/035355, filed Jun. 5, 2020, entitled “Passivated Silver Nanoparticle Coatings and Methods of Making the Same,” each of which are incorporated by reference herein in its entirety.

### STATEMENT REGARDING FEDERALLY SPONSORED RESEARCH

This invention was made with government support under 1449500 awarded by the National Science Foundation and 80NSSC19C0566 awarded by the National Aeronautical and Space Administration. The government has certain rights in the invention.

### BACKGROUND

### 1. Field of the Invention

The present disclosure relates generally to silver nanoparticles and methods of making and using such nanoparticles.

### 2. Description of Related Art

Silver is used as a biocide for disinfection and biofouling control in a variety of applications, including water treatment, medical supplies, medical devices, textiles, and the like. However, antimicrobial coatings containing silver nanoparticles have high leaching rates, leading to only short-term effectiveness, which increases both overall cost and the risk of biofilm development. While there has been exploration of in situ coatings of silver nanoparticles with a silver sulfide core-shell structure to decrease leaching rates, this is limited in application to coatings. Thus, there exists a need for silver nanoparticles with low leaching rates that are not formed in situ, allowing for more versatility with respect to applications.

### SUMMARY

The present invention provides among other things silver nanoparticles and methods of making the same. The nanoparticles may be sulfidated to decrease the silver leaching rate and sustain the biocidal properties. Such nanoparticles may be applied as a coating or additive to substrates such as metals, alloys, polymers, membranes, textiles, and other such materials, allowing for the substrates to exhibit antimicrobial properties.

In various embodiments, a nanoparticle may comprise a silver nanoparticle core, wherein the silver nanoparticle core may have a diameter of between approximately 3 nanometers (nm) and approximately 350 nm, and a plurality of silver sulfide (Ag₂S) molecules bonded to the silver nanoparticle core, wherein the plurality of silver sulfide molecules are arranged to form a partial shell around the silver nanoparticle core. In some embodiments, the weight percentage of sulfur may be between approximately 0.1% and approximately 7%.

In various embodiments, a method of producing nanoparticles may comprise making or obtaining a solution of silver nitrate (AgNO₃), making or obtaining a solution of a nucleating agent, and combining the solution of silver nitrate and the solution of the nucleating agent to form a suspension of silver nanoparticles. In some embodiments, the method may further comprise making or obtaining a solution of sodium nitrate (NaNO₃), adding a sulfidation agent to the solution of sodium nitrate to form a sulfide solution, and combining the suspension of silver nanoparticles and the sulfide solution to form a suspension of sulfidated silver nanoparticles (Ag/Ag₂S) having a core-shell structure. In some embodiments, the method may further comprise centrifuging the suspension of sulfidated silver nanoparticles, decanting the supernatant, and freeze drying the particles. In some embodiments, the nucleating agent may be selected from the group consisting of sodium borohydride, hydrazine, D-glucose, hyaluronic acid, and combinations thereof. In some embodiments, the nucleating agent is sodium borohydride. In some embodiments, the solution of silver nitrate and the solution of the nucleating agent may have a concentration from approximately 1 millimolar (mM) to approximately 300 mM. In some embodiments, the solution of silver nitrate and the solution of the nucleating agent may have approximately the same concentration. In some embodiments, the sulfidation agent may be selected from the group consisting of sodium sulfide, sodium thiosulfate, thiocarbamide, thioacetamide, and combinations thereof. In some embodiments, the sulfidation agent is sodium sulfide. In some embodiments, the solution of sodium nitrate and the sulfidation agent may have a concentration from approximately 10⁻¹ M to approximately 10⁻⁵ M. In some embodiments, the solution of sodium nitrate may have a concentration approximately ten times the concentration of the solution of the sulfidation agent.

In various embodiments, a method of producing nanoparticles may comprise making or obtaining a suspension of silver nanoparticles, making or obtaining a solution of sodium nitrate and a sulfidation agent, and combining the suspension of silver nanoparticles and the sulfide solution to form a suspension of sulfidated silver nanoparticles (Ag/Ag₂S) having a core-shell structure. In some embodiments, the sulfidation agent may be selected from the group consisting of sodium sulfide, sodium thiosulfate, thiocarbamide, thioacetamide, and combinations thereof. In some embodiments, the sulfidation agent is sodium sulfide. In some embodiments, the solution of sodium nitrate and the sulfidation agent may have a concentration from approximately 10⁻¹ M to approximately 10⁻⁵ M. In some embodiments, the solution of sodium nitrate has a concentration approximately ten times the concentration of the solution of the sulfidation agent.

In various embodiments, a method of producing nanoparticles may comprise making or obtaining a solution of silver nitrate (AgNO₃), making or obtaining a solution of D-glucose, combining the solution of silver nitrate and the solution of the nucleating agent to form a suspension of silver nanoparticles, making or obtaining a solution of sodium nitrate (NaNO₃), adding a solution of sodium sulfide to the solution of sodium nitrate to form a sulfide solution, and combining the suspension of silver nanoparticles and the sulfide solution to form a suspension of sulfidated silver nanoparticles (Ag/Ag₂S) having a core-shell structure. In some embodiments, the solution of silver nitrate may have a concentration of approximately 1 millimolar (mM). In some embodiments, the solution of D-glucose may have a concentration of approximately 3 mM. In some embodiments, the solution of sodium nitrate may have a concentration of approximately 10⁻² M. In some embodiments, the solution of sodium sulfide may have a concentration of approximately 10⁻³ M.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventor is fully aware that he can be his own lexicographer if desired. The inventor expressly elects, as his own lexicographers, to use only the plain and ordinary meaning of terms in the specification and claims unless he clearly states otherwise and then further, expressly sets forth the “special” definition of that term and explains how it differs from the plain and ordinary meaning. Absent such clear statements of intent to apply a “special” definition, it is the inventor's intent and desire that the simple, plain and ordinary meaning to the terms be applied to the interpretation of the specification and claims.

The inventor is also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventor is fully informed of the standards and application of the special provisions of 35 U.S.C. § 112(f). Thus, the use of the words “function,” “means” or “step” in the Detailed Description or Description of the Drawings or claims is not intended to somehow indicate a desire to invoke the special provisions of 35 U.S.C. § 112(f), to define the invention. To the contrary, if the provisions of 35 U.S.C. § 112(f) are sought to be invoked to define the inventions, the claims will specifically and expressly state the exact phrases “means for” or “step for, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventor not to invoke the provisions of 35 U.S.C. § 112(f). Moreover, even if the provisions of 35 U.S.C. § 112(f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

The foregoing and other aspects, features, and advantages will be apparent to those artisans of ordinary skill in the art from the DETAILED DESCRIPTION and DRAWINGS, and from the CLAIMS.

### BRIEF DESCRIPTION OF THE SEVERAL VIEWS OF THE DRAWINGS

A more complete understanding of the present invention may be derived by referring to the detailed description when considered in connection with the following illustrative figures. In the figures, like reference numbers refer to like elements or acts throughout the figures.

FIG. 1 depicts an exemplary core-shell structure of a sulfidated silver nanoparticle.

FIGS. 2A-2D depict transmission electron microscopy (TEM) images of a first batch of non-sulfidated silver nanoparticles on a substrate.

FIGS. 3A-3D depict TEM images of a second batch of non-sulfidated silver nanoparticles on a substrate.

FIG. 4A depicts an EDAX image of a first sulfidated silver nanoparticle.

FIG. 4B depicts an EDAX image of the Kα of silver atoms for the first sulfidated silver nanoparticle.

FIG. 4C depicts an EDAX image of the Kα of sulfur atoms for the first sulfidated silver nanoparticle.

FIG. 5 depicts a map sum spectrum for a first batch of sulfidated silver nanoparticles.

FIG. 6A depicts an EDAX image of a second sulfidated silver nanoparticle.

FIG. 6B depicts an EDAX image of the Kα of silver atoms for the second sulfidated silver nanoparticle.

FIG. 7 depicts a map sum spectrum for a second batch of sulfidated silver nanoparticles.

FIG. 8A depicts an EDAX image of a third sulfidated silver nanoparticle.

FIG. 8B depicts an EDAX image of the Kα of silver atoms for the third sulfidated silver nanoparticle.

FIG. 8C depicts an EDAX image of the Kα of sulfur atoms for the third sulfidated silver nanoparticle.

FIG. 9 depicts a map sum spectrum for a third batch of sulfidated silver nanoparticles.

Elements and acts in the figures are illustrated for simplicity and have not necessarily been rendered according to any particular sequence or embodiment.

### DETAILED DESCRIPTION

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. In many cases, a description of the operation is sufficient to enable one to implement the various forms of the invention, particularly when the operation is to be implemented in software. It should be noted that there are many different and alternative configurations, devices and technologies to which the disclosed inventions may be applied. The full scope of the inventions is not limited to the examples that are described below.

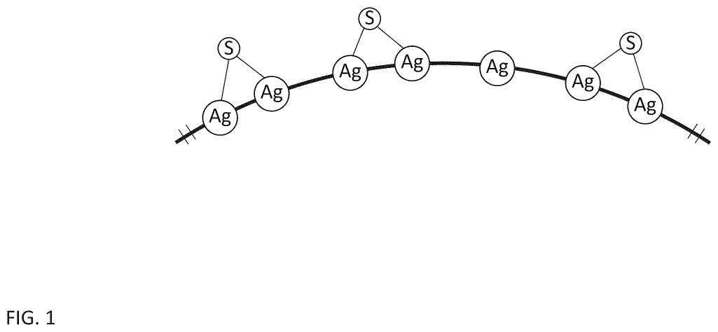

In one application, silver nanoparticles (Ag NPs) may be made in a solution or powder form to be used as additives or coatings, which provides for more versatility with respect to the applications compared to forming Ag NPs directly on a substrate. The Ag NPs may additionally have a silver sulfide (Ag₂S) shell to control the release of the Ag NPs, wherein the resulting particles have an Ag/Ag₂S core-shell structure. FIG. 1 illustrates an exemplary segment of the Ag/Ag₂S core-shell structure. In some embodiments, the AG NPs may have diameter range from approximately 3 nanometers (nm) to approximately 350 nm. For example, the Ag NPs may have a diameter of approximately 3 nm, approximately 4 nm, approximately 5 nm, approximately 6 nm, approximately 7 nm, approximately 8 nm, approximately 9 nm, approximately 10 nm, approximately 20 nm, approximately 30 nm, approximately 40 nm, approximately 50 nm, approximately 60 nm, approximately 70 nm, approximately 80 nm, approximately 90 nm, approximately 100 nm, approximately 110 nm, approximately 120 nm, approximately 130 nm, approximately 140 nm, approximately 150 nm, approximately 160 nm, approximately 170 nm, approximately 180 nm, approximately 190 nm, approximately 200 nm, approximately 210 nm, approximately 220 nm, approximately 230 nm, approximately 240 nm, approximately 250 nm, approximately 260 nm, approximately 270 nm, approximately 280 nm, approximately 290 nm, approximately 300 nm, approximately 310 nm, approximately 320 nm, approximately 330 nm, approximately 340 nm, approximately 350 nm, or any range between any two of these values, including endpoints. In some embodiments, the weight percentage of the sulfur may be from approximately 0% (non-sulfidated) to approximately 7%. For example, the weight percentage of the sulfur may be approximately 0%, approximately 0.1%, approximately 0.2%, approximately 0.3%, approximately 0.4%, approximately 0.5%, approximately 0.6%, approximately 0.7%, approximately 0.8%, approximately 0.9%, approximately 1%, approximately 2%, approximately 3%, approximately 4%, approximately 5%, approximately 6%, approximately 7%, or any range between any two of these values, including endpoints. The weight percentage of sulfur for a sulfidated Ag NP may be from approximately 0.1% to approximately 7%. In some examples, the weight percentage of the sulfur is approximately 1%.

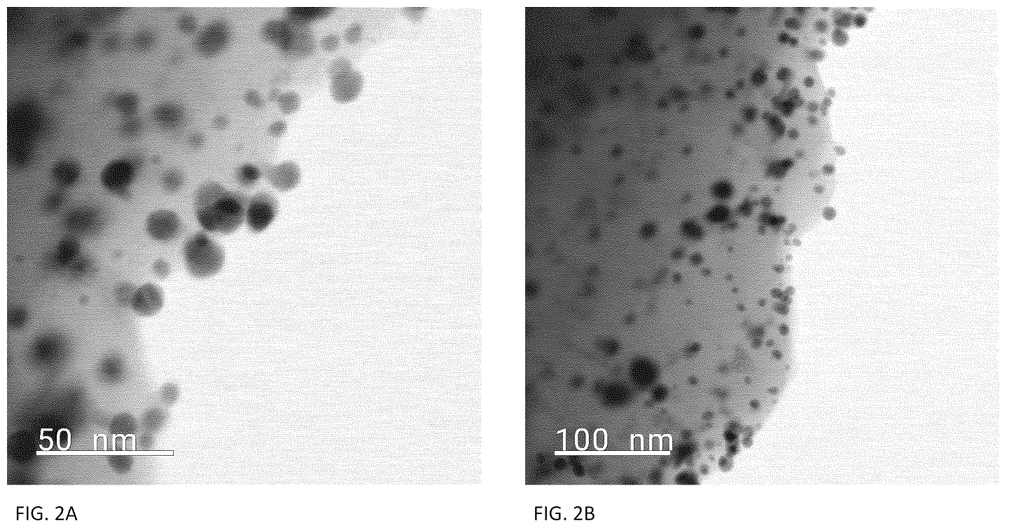

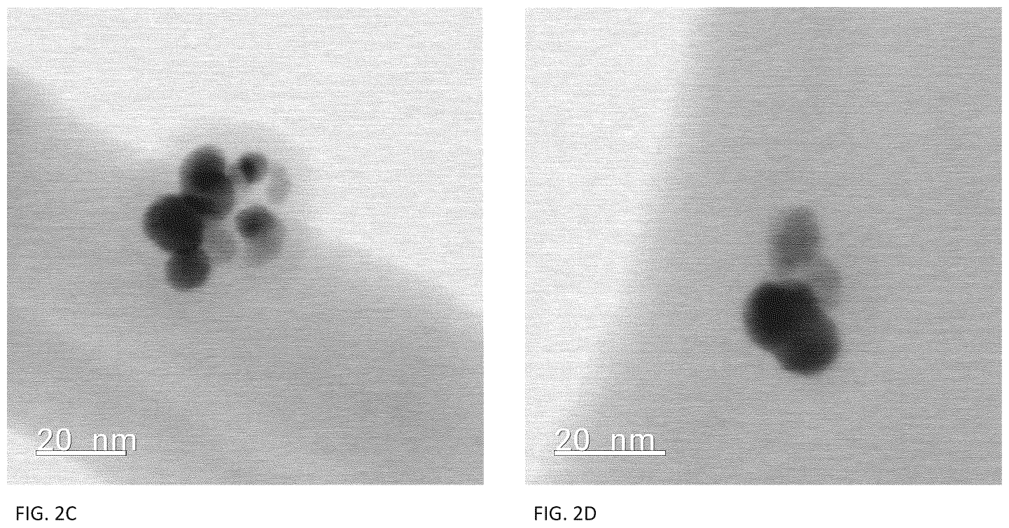

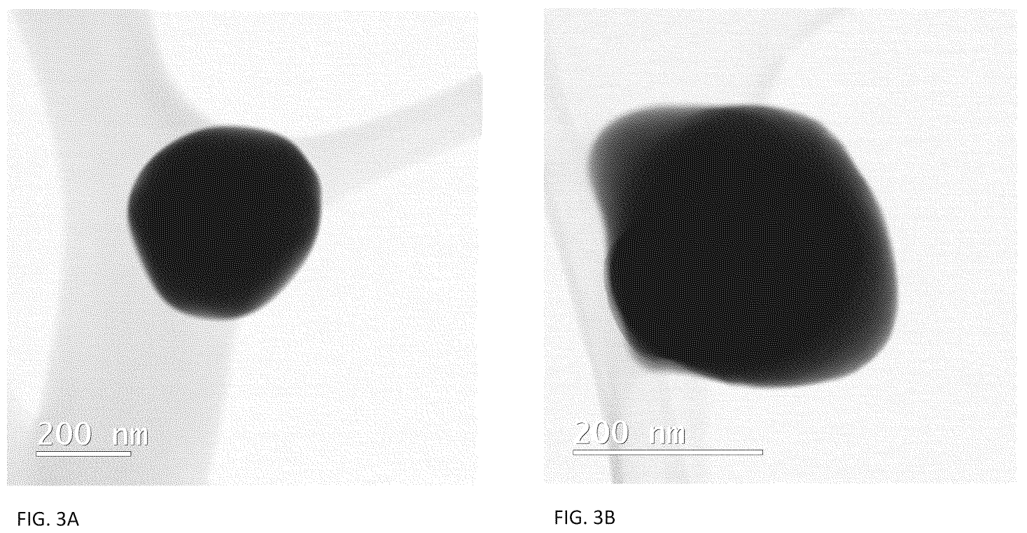

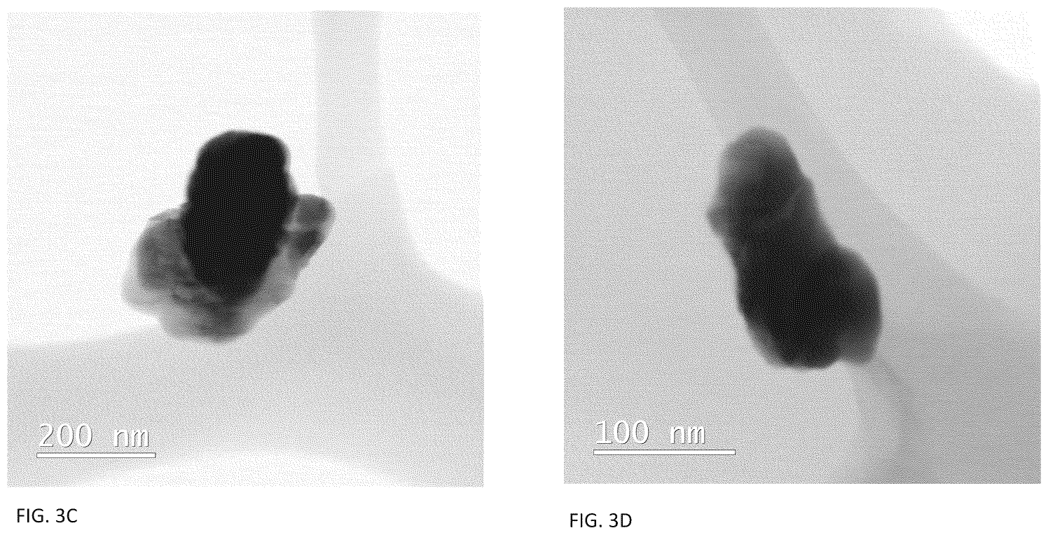

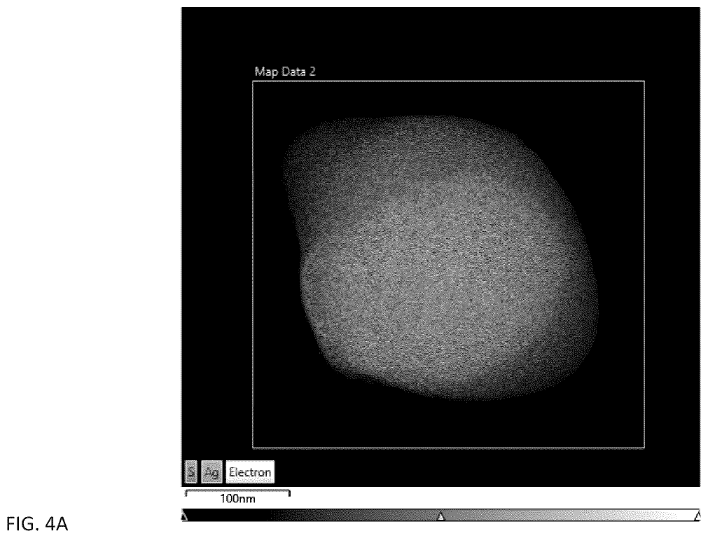

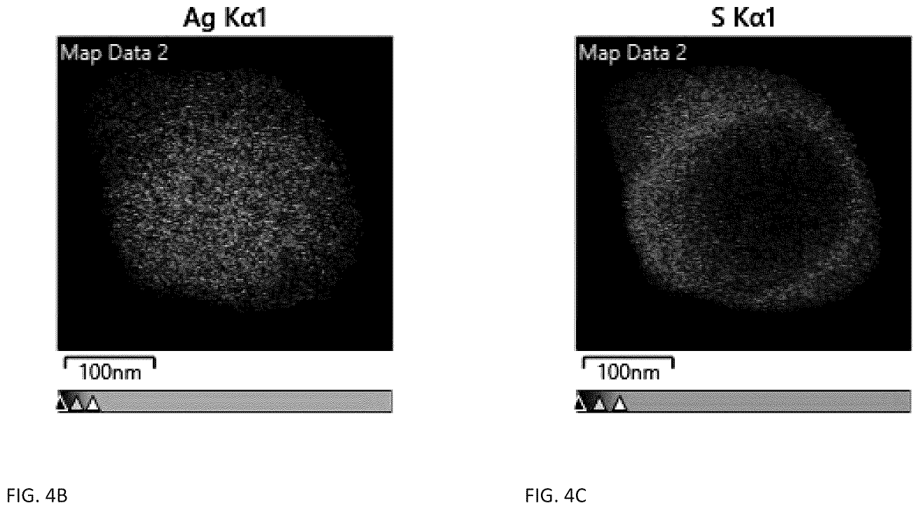

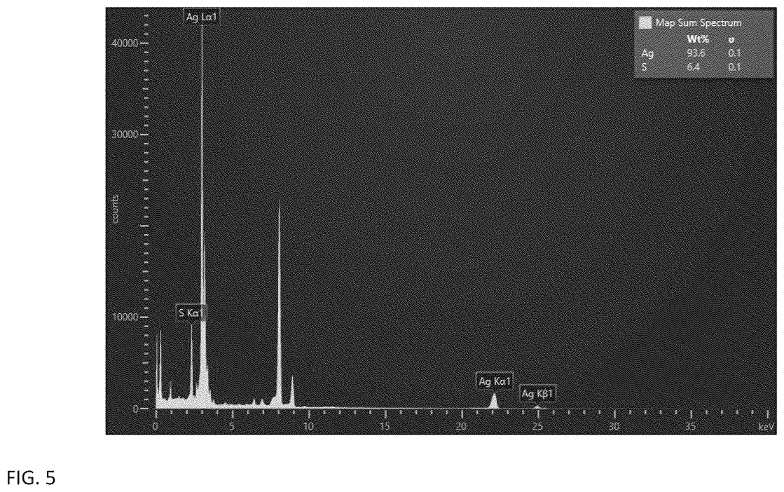

FIGS. 2A-2D are TEM images of a first batch of non-sulfidated Ag NPs produced using methods described herein. These images are zoomed out to show potential larger-order structures of the Ag NPs. The morphology of these Ag NPs is shown to have crystalline structures with mostly spherical shape. Some of these particles form aggregates. These particles have an average diameter of 8.1±2.2 nm and follow a Gaussian distribution with sizes ranging from 4.1 nm to 15 nm. FIGS. 3A-3D are TEM images of a second batch of non-sulfidated Ag NPs produced using methods described herein. These images are zoomed out to show potential larger-order structures of the Ag NPs. The morphology of these Ag NPs is shown to have crystalline structures with mostly spherical shape. Some of these particles form aggregates. These particles have an average diameter of 27.0±9.4 nm and follow a Gaussian distribution with sizes ranging from 11.56 nm to 47.02 nm. FIGS. 4A-4E are TEM images of a first batch of sulfidated Ag NPs produced using methods described herein. These images are zoomed out to show potential larger-order structures of the Ag NPs. The morphology of these Ag NPs is shown to have crystalline structures with mostly spherical shape. Some of these particles form aggregates. These particles have an average diameter of 10.3±2.8 nm and follow a Gaussian distribution with sizes ranging from 5.21 nm to 17.1 nm. FIGS. 5A-5E are TEM images of a second batch of sulfidated Ag NPs produced using methods described herein. These images are zoomed in to show individual Ag NPs. These particles are larger than 100 nm with diameters up to approximately 350 nm, showing significant variation from the Ag NPs in FIGS. 4A-4E. Thus, the size of the Ag NPs may be controlled using variations in production.

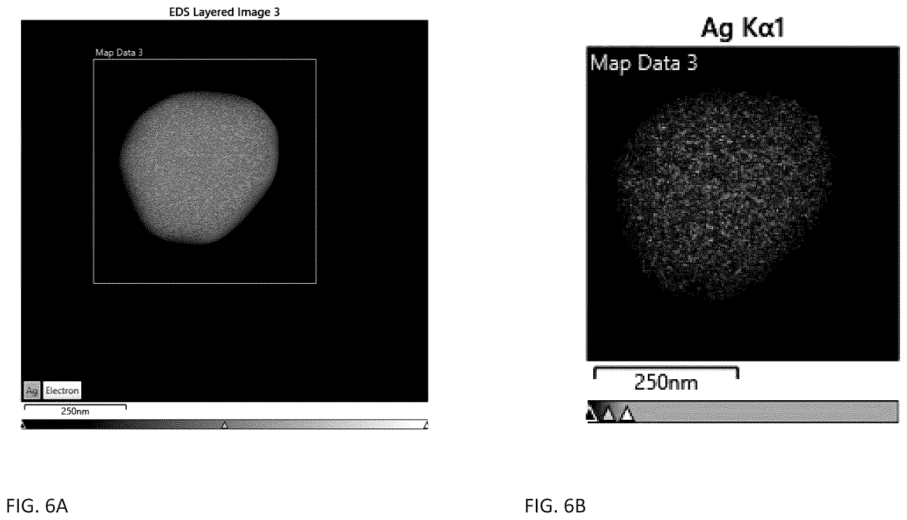

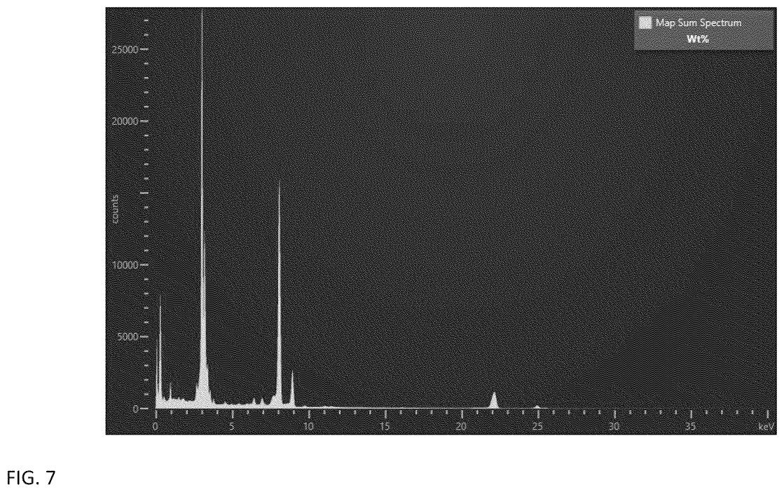

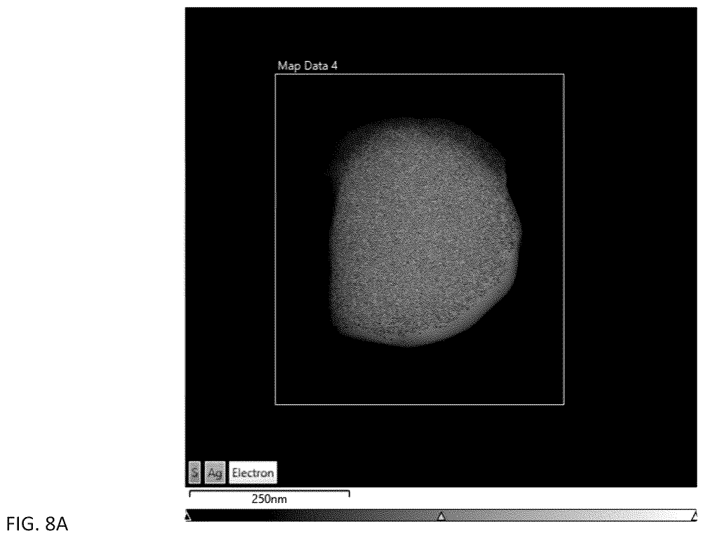

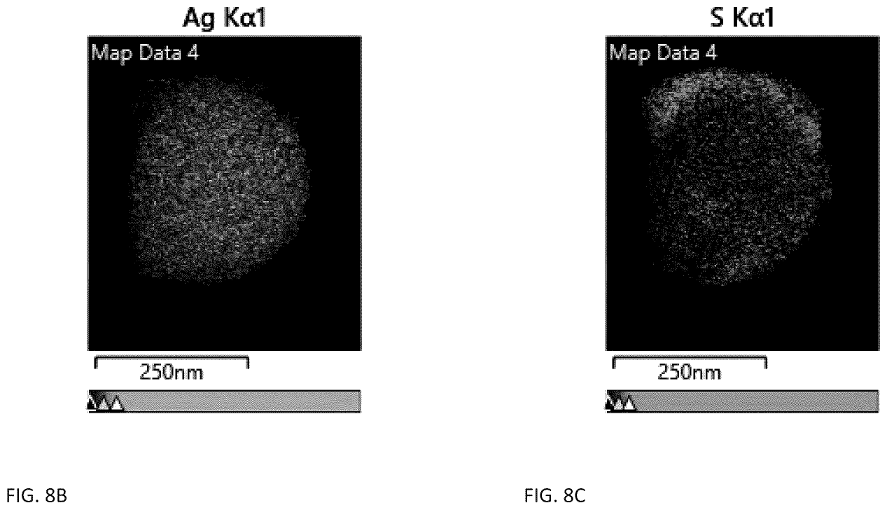

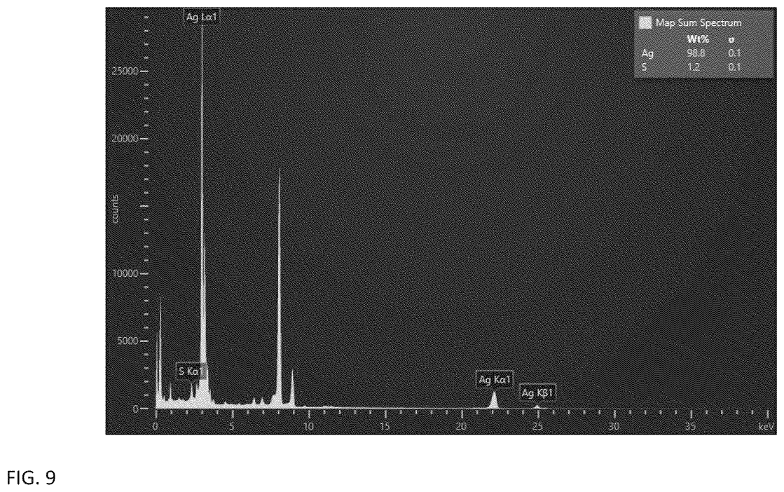

FIGS. 6A-6C are energy-dispersive x-ray spectroscopy (EDAX) images of a first sulfidated Ag NP. FIG. 6A shows the presence of both silver and sulfur in the particle. FIG. 6B shows just the silver in the particle, which is concentrated in the center of the particle. FIG. 6C shows just the sulfur in the particle, which is concentrated in the outer shell of the particle. These images exemplify the Ag/Ag₂S core-shell structure of the particle and that there are no other elements present in the particle. FIG. 7 is a map sum spectrum for a first batch of sulfidated Ag NPs. This shows the dispersion of weight percentages of silver and sulfur of the particles in the batch. Overall, silver has a weight percentage of 91.6% and sulfur has a weight percentage of 6.4%. FIGS. 8A-8B are EDAX images of a second sulfidated Ag NP. FIG. 8A shows the silver in a particle with the particle visible. FIG. 8B shows just the silver in the particle, which is evenly distributed in the particle. FIG. 9 is a map sum spectrum for a second batch of sulfidated Ag NPs. This shows very similar results to the map sum spectrum of FIG. 7 with some mild variations left of the primary apex. Overall, silver has a weight percentage of 98.8% and sulfur has a weight percentage of 1.2%. This shows mild variance from the map sum spectrum of FIG. 7 and FIG. 9. Overall, these TEM and EDAX images illustrate that the size and composition of the ag NPs may be tailored to specific needs depending on the process specification. Methods of producing these nanoparticles are described herein.

In various embodiments, a solution of a nucleating agent may be combined with a solution of silver nitrate (AgNO₃) to produce a solution of silver nanoparticles. In some embodiments, the solution of silver nitrate may have a concentration from approximately 1 millimolar (mM) to approximately 300 mM. For example, the solution of silver nitrate may have a concentration of approximately 1 mM, approximately 3 mM, approximately 5 mM, approximately 10 mM, approximately 20 mM, approximately 30 mM, approximately 40 mM, approximately 50 mM, approximately 60 mM, approximately 70 mM, approximately 80 mM, approximately 90 mM, approximately 100 mM, approximately 110 mM, approximately 120 mM, approximately 130 mM, approximately 140 mM, approximately 150 mM, approximately 160 mM, approximately 170 mM, approximately 180 mM, approximately 190 mM, approximately 200 mM, approximately 210 mM, approximately 220 mM, approximately 230 mM, approximately 240 mM, approximately 250 mM, approximately 260 mM, approximately 270 mM, approximately 280 mM, approximately 290 mM, approximately 300 mM, or any range between any two of these values, including endpoints. In some embodiments, the solution of silver nitrate has a concentration of approximately 150 mM. In some embodiments, the nucleating agent may be sodium borohydride (NaBH₄), hydrazine (N₂H₄), D-glucose (C₆H₁₂O₆), hyaluronic acid (C14H21NO₁₁), or any other suitable nucleating agent for producing silver nanoparticles. In some embodiments, the nucleating agent is sodium borohydride. In some embodiments, the solution of the nucleating agent may have a concentration from approximately 1 mM to approximately 300 mM. For example, the solution of the nucleating agent may have a concentration of approximately 1 mM, approximately 3 mM, approximately 5 mM, approximately 10 mM, approximately 20 mM, approximately 30 mM, approximately 40 mM, approximately 50 mM, approximately 60 mM, approximately 70 mM, approximately 80 mM, approximately 90 mM, approximately 100 mM, approximately 110 mM, approximately 120 mM, approximately 130 mM, approximately 140 mM, approximately 150 mM, approximately 160 mM, approximately 170 mM, approximately 180 mM, approximately 190 mM, approximately 200 mM, approximately 210 mM, approximately 220 mM, approximately 230 mM, approximately 240 mM, approximately 250 mM, approximately 260 mM, approximately 270 mM, approximately 280 mM, approximately 290 mM, approximately 300 mM, or any range between any two of these values, including endpoints. In some embodiments, the solution of the nucleating agent has a concentration of approximately 150 mM. In some embodiments, the solution of silver nitrate and the solution of the nucleating agent have approximately the same concentration. The solutions may be stirred for a time period of from approximately 1 hour to approximately 36 hours. For example, the time period may be approximately 1 hour, approximately 2 hours, approximately 3 hours, approximately 4 hours, approximately 5 hours, approximately 6 hours, approximately 7 hours, approximately 8 hours, approximately 9 hours, approximately 10 hours, approximately 11 hours, approximately 12 hours, approximately 13 hours, approximately 14 hours, approximately 15 hours, approximately 16 hours, approximately 17 hours, approximately 18 hours, approximately 19 hours, approximately 20 hours, approximately 21 hours, approximately 22 hours, approximately 23 hours, approximately 24 hours, approximately 25 hours, approximately 26 hours, approximately 27 hours, approximately 28 hours, approximately 29 hours, approximately 30 hours, approximately 31 hours, approximately 32 hours, approximately 33 hours, approximately 34 hours, approximately 35 hours, approximately 36 hours, or any range between any two of these values, including endpoints. In some embodiments, the solutions are stirred for a time period of approximately 24 hours. The solutions may be stirred at room temperature of at higher temperatures depending on the nucleating agent used.

A sulfide solution may be added to form the silver sulfide shell. The sulfide solution may be formed by combining a solution of sodium nitrate (NaNO₃) and adding a sulfidation agent. In some embodiments, the solution of sodium nitrate may have a concentration from approximately 10⁻¹ M to approximately 10⁻⁵ M. For example, the solution of sodium nitrate may have a concentration of approximately 10⁻¹ M, approximately 10⁻² M, approximately 10⁻³ M, approximately 10⁻⁴ M, approximately 10⁻⁵ M, or any range between any two of these values, including endpoints. In some embodiments, the solution of sodium nitrate has a concentration of approximately 10⁻² M. In some embodiments, the sulfidation agent may be sodium sulfide (Na₂S), sodium thiosulfate (Na₂S₂O₃), thiocarbamide (SC(NH₂)₂, thioacetamide (C₂H₅NS), or combinations thereof. In some embodiments, the sulfidation agent is sodium sulfide. In some embodiments, the sulfidation agent may have a concentration from approximately 10⁻¹ M to approximately 10⁻⁵ M. For example, the sulfidation agent may have a concentration of approximately 10⁻¹ M, approximately 10⁻² M, approximately 10⁻³ M, approximately 10⁻⁴ M, approximately 10⁻⁵ M, or any range between any two of these values, including endpoints. In some embodiments, the sulfidation agent has a concentration of approximately 10⁻³ M. The sulfide solution may then be mixed with the solution of silver nanoparticles. In some embodiments, when mixing the sulfide solution with the solution of silver nanoparticles, the volume ratio of the solution of silver nanoparticles to the sulfide solution may be from approximately 4:1 to approximately 14:1. For example, the volume ratio of the solution of silver nanoparticles to the sulfide solution may be approximately 4:1, approximately 5:1, approximately 6:1, approximately 7:1, approximately 8:1, approximately 9:1, approximately 10:1, approximately 11:1, approximately 12:1, approximately 13:1, approximately 14:1, or any range between any two of these values, including endpoints. In some embodiments, the volume ratio of the solution of silver nanoparticle to the sulfide solution is approximately 9:1. The solutions may be stirred for a time period of from approximately 0.5 hour to approximately 24 hours. For example, the time period may be approximately 0.5 hour, approximately 1 hours, approximately 2 hours, approximately 3 hours, approximately 4 hours, approximately 5 hours, approximately 6 hours, approximately 7 hours, approximately 8 hours, approximately 9 hours, approximately 10 hours, approximately 11 hours, approximately 12 hours, approximately 13 hours, approximately 14 hours, approximately 15 hours, approximately 16 hours, approximately 17 hours, approximately 18 hours, approximately 19 hours, approximately 20 hours, approximately 21 hours, approximately 22 hours, approximately 23 hours, approximately 24 hours, or any range between any two of these values, including endpoints. In some embodiments, the solutions are stirred for a time period of approximately 24 hours. To produce non-sulfidated Ag NPs, the sulfide solution may be omitted.

After being produced, the Ag NPs may be washed. In some embodiments, the washing process may include allowing the Ag NP solution to settle, removing the liquid solution, rinsing the Ag NPs with deionized (DI) water, or any combination thereof. DI water may then be added to the Ag NPs to form a solution.

To make a powder form of the Ag NPs the Ag NP solution may be centrifuged and freeze dried. In some embodiments, the Ag NP solution may be centrifuged for a time period from approximately 10 minutes to approximately 60 minutes. For example, the time period may be approximately 10 minutes, approximately 15 minutes, approximately 20 minutes, approximately 25 minutes, approximately 30 minutes, approximately 35 minutes, approximately 40 minutes, approximately 45 minutes, approximately 50 minutes, approximately 55 minutes, approximately 60 minutes, or any range between any two of these values, including endpoints. In some embodiments, the supernatant may be removed. In some embodiments, the Ag NPs are freeze dried for a time period from approximately 1 hour to approximately 36 hours. For example, the time period may be approximately 1 hour, approximately 2 hours, approximately 3 hours, approximately 4 hours, approximately 5 hours, approximately 6 hours, approximately 7 hours, approximately 8 hours, approximately 9 hours, approximately 10 hours, approximately 11 hours, approximately 12 hours, approximately 13 hours, approximately 14 hours, approximately 15 hours, approximately 16 hours, approximately 17 hours, approximately 18 hours, approximately 19 hours, approximately 20 hours, approximately 21 hours, approximately 22 hours, approximately 23 hours, approximately 24 hours, approximately 25 hours, approximately 26 hours, approximately 27 hours, approximately 28 hours, approximately 29 hours, approximately 30 hours, approximately 31 hours, approximately 32 hours, approximately 33 hours, approximately 34 hours, approximately 35 hours, approximately 36 hours, or any range between any two of these values, including endpoints. In some embodiments, the Ag NPs are freeze dried for a time period approximately 24 hours. In some embodiments, the particles may be freeze dried at a temperature from approximately −20° C. to approximately −100° C. For example, the temperature may be approximately −20° C., approximately −30° C., approximately −40° C., approximately −50° C., approximately −60° C., approximately −70° C., approximately −80° C., approximately −90° C., approximately −100° C., or any range between any two of these values, including endpoints.

The Ag NPs may be applied to substrates as a coating or mixed with substrates as an additive to allow the substrates to exhibit antimicrobial properties. Examples of substrates may include metals, alloys, polymers, membranes, textiles, and other such materials or combinations thereof.

### EXAMPLES

### Example 1: Making Non-Sulfidated Silver Nanoparticles Using Sodium Borohydride

Non-sulfidated Ag NPs may be made starting with solid silver nitrate, solid sodium borohydride, and DI water. The silver nitrate solution may be made by dissolving the solid silver nitrate into approximately 800 mL of DI water to create a silver nitrate solution that has a concentration of approximately 150 mM. The solid sodium borohydride (nucleating agent) may be dissolved into approximately 100 mL of DI water to create a sodium borohydride solution that has a concentration of approximately 150 mM. These aqueous solutions may then be mixed by adding the sodium borohydride solution to the silver nitrate solution in a dropwise fashion. The resulting solution may be stirred for approximately 24 hours to form an aqueous suspension of Ag NPs. The Ag NPs may then be washed. The washing may include removing the liquid portion and rinsing the Ag NPs with DI water. This step may be performed multiple times. 1 L of DI water may then be added to the Ag NPs to create an Ag NP suspension. The Ag NP suspension may be used as a coating or additive, or it may be further processed into a powder. To create the powder, the suspension may be centrifuged for approximately 30 minutes. The supernatant may then be decanted, and the Ag NPs may be rinsed. The Ag NPs may be freeze dried for approximately 24 hours at approximately −50° C. The resulting powder may be used as a coating or additive.

### Example 2: Making Sulfidated Silver Nanoparticles Using Sodium Borohydride

Sulfidated Ag NPs may be made by performing the steps described in Example 1 up to the washing process to create the suspension of Ag NPs. Sodium nitrate may be dissolved in 100 mL of DI water to create a solution with a sodium nitrate concentration of approximately 10⁻² M. Sodium sulfide (sulfidation agent) may be dissolved in the sodium nitrate solution to create a sulfide solution with a sodium sulfide concentration of approximately 10⁻³ M. The solution may then be added to the Ag NP suspension in a dropwise fashion and stirred for approximately 24 hours. The resulting suspension may be washed as described in Example 1 to form a sulfidated Ag NP suspension that can be used as a coating or additive. The same process as described in Example 1 may be used to create a powder that can be used as a coating or additive.

### Example 3: Making Non-Sulfidated Silver Nanoparticles Using D-Glucose

Non-sulfidated Ag NPs may be made starting with solid silver nitrate, D-glucose powder, and DI water. The silver nitrate solution may be made by dissolving the solid silver nitrate into approximately 400 mL of DI water to create a silver nitrate solution that has a concentration of approximately 1 mM. D-glucose powder (nucleating agent) may be dissolved into 100 mL of DI water to create a D-glucose solution that has a concentration of approximately 3 mM. These two solutions may then be mixed for approximately 90 minutes at approximately 95° C. to form an aqueous suspension of Ag NPs. The resulting suspension may be washed as described in Example 1 to form an Ag NP suspension that can be used as a coating or additive. The same process as described in Example 1 may be used to create a powder that can be used as a coating or additive.

### Example 4: Making Sulfidated Silver Nanoparticles Using D-Glucose

Sulfidated Ag NPs may be made by starting with solid silver nitrate, D-glucose powder, and DI water. The silver nitrate solution may be made by dissolving the solid silver nitrate into approximately 150 mL of DI water to create a silver nitrate solution that has a concentration of approximately 1 mM. D-glucose powder (nucleating agent) may be dissolved into 100 mL of DI water to create a D-glucose solution that has a concentration of approximately 3 mM. These two solutions may then be mixed for approximately 90 minutes at approximately 95° C. to form an aqueous suspension of Ag NPs. Sodium nitrate may be dissolved in 250 mL of DI water to create a solution with a sodium nitrate concentration of approximately 10⁻² M. Sodium sulfide (sulfidation agent) may be dissolved in the sodium nitrate solution to create a sulfide solution with a sodium sulfide concentration of approximately 10⁻³ M. The sulfide solution may be added to the suspension of Ag NPs and stirred for approximately 24 hours. The resulting suspension may be washed as described in Example 1 to form an Ag NP suspension that can be used as a coating or additive. The same process as described in Example 1 may be used to create a powder that can be used as a coating or additive.

### Example 5: Antimicrobial Effectiveness of the Silver Nanoparticles

Table 1 below shows the percentage of bacteria that were inactivated by surfaces coated with both non-sulfidated and sulfidated Ag NPs.

TABLE 1

| | | | Loading | | Inactivation |
|---|---|---|---|---|---|
| Surface | Coating | Chemistry | (ng/cm²) | Strain | (%) |
| Membrane | Non-Sulfidated | NaBH₄ | 2800 | P. aeruginosa | 60.5 |
| Membrane | Non-Sulfidated | NaBH₄ | 2800 | E. coli | 50 |
| Membrane | Sulfidated | NaBH₄ | 2733.33 | P. aeruginosa | 56 |
| Membrane | Sulfidated | NaBH₄ | 2733.33 | E. coli | 57.1 |
| SS 444 Coupon | Non-Sulfidated | NaBH₄ | 159.74 | P. aeruginosa | 39.1 |
| SS 444 Coupon | Sulfidated | NaBH₄ | 46.15 | P. aeruginosa | 69.7 |

This data shows significant bacteria inactivation across different surfaces, types of Ag NPs, and bacteria strains, though there are variations. The effectiveness for both the non-sulfidated and sulfidated Ag NPs shows slight variations across strains of bacteria and type of Ag NP when applied to membranes. However, the effectiveness of the sulfidated Ag NPs appears to be significantly higher than the effectiveness of the non-sulfidated Ag NPs when applied to stainless steel using the bacteria P aeruginosa.

We claim:

1. A nanoparticle comprising:
a silver nanoparticle core; and
a plurality of silver sulfide (Ag2S) molecules bonded to the silver nanoparticle core, wherein the plurality of silver sulfide molecules are arranged to form a partial shell around the silver nanoparticle core.

2. The nanoparticle as recited in claim 1, wherein the weight percentage of sulfur is between approximately 0.1% and approximately 7% and the silver nanoparticle core has a diameter of between approximately 3 nanometers (nm) and approximately 350 nm.

4. A method of producing nanoparticles comprising:
A. making or obtaining a solution of silver nitrate (AgNO₃);
B. making or obtaining a solution of a nucleating agent; and
C. combining the solution of silver nitrate and the solution of the nucleating agent to form a suspension of silver nanoparticles.

5. The method of claim 4, further comprising:
A. making or obtaining a solution of sodium nitrate (NaNO₃);
B. adding a sulfidation agent to the solution of sodium nitrate to form a sulfide solution; and
C. combining the suspension of silver nanoparticles and the sulfide solution to form a suspension of sulfidated silver nanoparticles (Ag/Ag₂S) having a core-shell structure.

6. The method of claim 5, further comprising:
centrifuging the suspension of sulfidated silver nanoparticles;
decanting the supernatant; and
freeze drying the particles.

7. The method of claim 4, wherein:
A. the nucleating agent (Component B) is selected from the group consisting of sodium borohydride, hydrazine, D-glucose, hyaluronic acid, and combinations thereof;
B. a stabilizer (Component C) may be used by selecting from the group consisting of Polyvinylpyrrolidone (PVP), sodium citrate dihydrate, and combinations thereof; and
C. the solution of silver nitrate (Component A) and the solution of the nucleating agent (Component B) have a concentration from approximately 1 millimolar (mM) to approximately 300 mM;
D. the nucleating agent is sodium borohydride;
E. the solution of silver nitrate and the solution of the nucleating agent have approximately the same concentration;
F. the sulfidation agent (Component B) is selected from the group consisting of sodium sulfide, sodium thiosulfate, thiocarbamide, thioacetamide, and combinations thereof; and
G. the solution of sodium nitrate (Component A) and the sulfidation agent (Component B) have a concentration from approximately 10⁻¹ M to approximately 10⁻⁵ M; and
H. the solution of sodium nitrate has a concentration approximately ten times the concentration of the solution of the sulfidation agent.

8. A method of producing nanoparticles comprising:
A. making or obtaining a solution of silver nitrate (AgNO₃);
B. making or obtaining a solution of D-glucose as a nucleating agent;
C. combining the solution of silver nitrate and the solution of the nucleating agent to form a suspension of silver nanoparticles;
D. making or obtaining a solution of sodium nitrate (NaNO₃);
E. adding a solution of sodium sulfide to the solution of sodium nitrate to form a sulfide solution; and
F. combining the suspension of silver nanoparticles and the sulfide solution to form a suspension of sulfidated silver nanoparticles (Ag/Ag₂S) having a core-shell structure

9. The method of claim 8 Component F, wherein:
A. a stabilizer may be used by selecting from the group consisting of Polyvinylpyrrolidone (PVP), sodium citrate dihydrate, and combinations thereof;
B. the solution of silver nitrate (Component A) has a concentration of approximately 1 millimolar (mM);
C. the solution of D-glucose (Component B) has a concentration of approximately 1 mM;
D. the solution of sodium nitrate (Component D) has a concentration of approximately 10⁻² M; and
E. the solution of sodium sulfide (Component E) has a concentration of approximately 10⁻³ M.
